(19) United States
(12) Patent Application Publication
(10) Pub. No.: US 2003/0087885 A1
(43) Pub. Date: May 8, 2003

(54) Pharmaceutical composition in the form of a gel or a solution based on dihydrotestosterone, process for preparing it and uses thereof

(76) Inventors: Valerie Masini-Eteve, Verrieres Le Buisson (FR); Brigitte Taravella, Paris (FR)

Correspondence Address: PIPER RUDNICK, P. O. BOX 64807, CHICAGO, IL, 60664-0807, US

(21) Appl. No.: 10/099,725

(22) Filed: Mar. 13, 2002

(30) Foreign Application Priority Data
Nov. 7, 2001 (EP) 01403166.0

(51) Int. Cl. A61K 31/57; A61K 9/70

(52) U.S. Cl. 514/177; 424/449

(57) ABSTRACT

The present invention relates to a pharmaceutical composition in the form of a gel or a solution and is characterized in that it contains dihydrotestosterone and also at least one penetration promoter, to processes for preparing it and to uses thereof.

The present invention relates to a pharmaceutical composition in the form of a gel or a solution based on dihydrotestosterone (DHT). The invention also relates to processes for preparing these formulations, as well as to their uses.

DHT is a metabolite of testosterone. In the sexual organs such as the prostate and the seminal vesicles, testosterone is reduced to DHT by an enzyme, 5-alpha reductase.

During andropause (or “male menopause” or “partial deficiency of ageing male”), the secretion of androgens decreases and, in certain cases, this may entail pathological disorders. In particular, a change in protein synthesis and in the enzymatic activities of the target tissues is observed. Added to the anomalies of testosterone production and transportation, are anomalies of metabolism by the target tissues, these anomalies to a large extent remaining undetected in the plasmatic assays usually performed and which form the specificity of the hypogonadism of the ageing male.

Thus, in an andropausal ageing male, testosterone is secreted in reduced amounts and above all it is insufficiently metabolized into DHT by the target tissues, as a result of a deficiency in 5-alpha-reductase, which is reflected by a reduction in androgenic activity. On the other hand, the aromatasic activity increases uniformly with age in the majority of men, which tends to maintain oestradiol level in the blood despite the fall in testosterone. The importance of these changes in intratissular activity is demonstrated by an increase in the level of SHBG (Serum Hormone Binding Globulin).

DHT is among the hormones required for the development of the male genital organs (penis, scrotum, prostate, seminal vesicles). It also plays a role in the development of male secondary sexual characteristics, namely pilosity, the development of musculature, the deepening of the voice and the appearance of libido. It also has an anabolizing action on the skeleton and a stimulatory action on the haematopoietic marrow at high dose.

DHT is prescribed in men as a systemic treatment for general androgenic deficiencies occurring as a result of a permanent hypogonadism of testicular or hypophyseal origin, or of a functional hypogonadism, usually due to surgical interventions, multiple injuries, bums or intense and sustained physical or psychological constraints.

DHT is also used as a local treatment in men in the case of gynaecomasty and balano-preputial sclero-atrophic lichen. It may also be prescribed in women in the case of vulval sclero-atrophic lichen.

The Applicant Company has already developed and marketed a DHT-based gel. This formulation forms the subject of a patent FR 2 519 252. The DHT concentration in this formulation is 2.5%.

The Applicant Company is also the proprietor of a patent EP 0 700 293 concerning the use of DHT in androgen therapy, and more particularly claiming the favourable effects of DHT on prostate hyperplasia. In the said patent, it is simply indicated that an aqueous-alcoholic gel having a DHT content of from 0.5% to 3.5% may be used. This being stated, the said patent gives no information regarding the constituents of such a gel, or regarding any method of manufacture, or regarding its efficacy, in particular in the case of a content that is very much lower than that usually used of 2.5%. Thus, to the Applicant Company's knowledge, no DHT-based gels, with a DHT content of less than 2.5%, were known to exist.

The gel applications are generally carried out once a day, either in the morning or in the evening, by spreading the gel liberally over a large surface of the skin: arms, shoulders, chest, abdomen or thighs, and then leaving it to dry for about five minutes before putting any clothing on the application area.

As regards the formulation in the form of a solution, it may be packaged in the form of a spray which would be readily vaporized over a large surface area of skin.

This involves a relatively large amount of gel or solution, namely 5 to 10 grams per day, depending on the therapeutic indication. The individual dosage control must also take account of the intensity of the androgen deficiency to be compensated for and also the tolerability.

It would thus be advantageous to be able to reduce the amount of gel or of solution to be applied and thus to reduce the application surface. This may also lead to an improvement in the patient's compliance with the treatment and to a reduction in the risk of cross-contamination between two individuals.

The secondary effects of DHT are far from being benign: irritability, psychomotor excitation, increase in weight, seborrhoea and acne, in men; there is also the problem of virilization in women.

In addition, as for any hormone, it is in the patients' interest to be able to reduce the dosage to the minimum effective amount, so as to reduce the adverse effects.

The Applicant Company has also found that the gel formulation according to patent FR 2 519 252 containing DHT at a rate of 2.5 g per 100 g of gel suffers from other drawbacks such as a relative physicochemical instability which may be reflected by the appearance of crystals in the gel during storage at room temperature.

The Applicant Company has thus sought to develop a novel DHT-based formulation allowing the concentration of DHT to be applied to the skin to be reduced significantly, while at the same time maintaining identical levels of skin absorption and efficiency of the product.

This provides an economic advantage, due to the reduction in the concentration of DHT used in the formulations, as well as the above-mentioned therapeutic advantages and advantages in terms of the physicochemical stability of the formulation.

The Applicant Company has, to its credit, developed a DHT-based pharmaceutical form for transdermal application for the treatment of the ageing male. In fact, the use of DHT in the treatment of hypogonadism in the ageing male presenting an abnormally high level of SHBG (sign of an intratissular dysfunction) is recommended.

In these men, the activity of the enzyme 5α-reductase is reduced. As a result, testosterone is poorly reduced to DHT, the active metabolite. It is thus more advantageous to treat ageing males directly using the naturally active androgen: DHT.

It is important to note that the estrogenic effects of testosterone are not manifested on the bone or on the majority of the other targets. Furthermore, testosterone is potentially harmful to the prostate since the increase in testosteronaemia is suspected of elevating the risk of cancer in the ageing male (Ly L. P. et al., J. Clin. Endocrinol Metab., 2001; 86: 4078-4088; Wang C. et al., J. Clin. Endocrinol Metab., 1998; 83: 2749-2757; Shaneyfelt T. et al., J. Clin. Oncol. 2000; 18: 847-853).

Thus, the invention relates to a pharmaceutical composition in the form of a gel or a solution, characterized in that it contains dihydrotestosterone and also at least one percutaneous absorption promoter.

The expression “percutaneous absorption promoter” means any molecule promoting the reversible diffusion of an active principle through the skin reversibly, and any solubilizing agent promoting the partition of the active principle from the vehicle to the horny layer of the epidermis.

According to one advantageous embodiment of the pharmaceutical composition according to the invention, the DHT content is less than 2.5%, preferably less than 1.5% and even more preferably is 0.7%, this percentage being expressed by weight per 100 g of formulation.

The pharmaceutical composition according to the invention also contains a solvent such as 95% ethanol, in a content of between 30% and 85%, preferably between 40% and 75% and even more preferably of 71%, these percentages being expressed by weight relative to 100 g of formulation.

The solvent used is a non-aqueous solvent capable of dissolving DHT and the absorption promoter. It will be chosen from compounds with a low boiling point, that is to say less than 100° C. at atmospheric pressure, so that it can evaporate rapidly on contact with the skin. Such solvents may be selected, alone or in combination, from volatile compounds such as ethanol, isopropanol or ethyl acetate; preferably ethanol and/or isopropanol. However, ethanol represents a preferred solvent according to the invention since it contributes with efficiency towards the transcutaneous passage of the active principle by evaporating rapidly on contact with the skin.

In order to achieve an effective concentration of active principle without, however, covering an excessively large area of skin, the active principle is combined with a percutaneous absorption promoter. The said promoter is introduced into the composition of the invention in a proportion of from 0.1% to 10%, preferably between 0.3% and 5% and even more preferably of 0.7%, these percentages being expressed by weight per 100 g of formulation.

This absorption promoter is chosen so as to improve the systemic passage of the DHT and thus to obtain the desired effects by means of an acceptable cutaneous coverage, that is to say less than 15 μg of DHT per cm², preferably 10 μg of DHT per cm² and even more preferably 7 μg of DHT per cm².

This cutaneous absorption promoter will be selected from substances that are compatible with the chosen non-aqueous solvent. Preferably, it will be chosen from the compounds mentioned below which have a necessary degree of solubility in the solvent under consideration and are non-irritant, non-allergenic and non-toxic. The chosen promoter will also have to be compatible with all of the components of the formulation selected and must be chemically and physically stable.

As examples of promoters that may be used, alone or in combination, in the pharmaceutical composition according to the invention and which have shown good properties in promoting the cutaneous absorption of active substances, mention may be made of: aliphatic fatty acid esters such as isopropyl myristate; fatty acids such as oleic acid; alcohols or polyols such as ethanol, propylene glycol and polyethylene glycols; components of essential oils and terpine derivatives (such as eugenol, geraniol, nerol, eucalyptol or menthol); surfactants; moisturizers such as glycerol, urea; keratolytic agents such as α-hydroxy acids.

Isopropyl myristate represents the preferred absorption promoter for the pharmaceutical composition according to the invention.

When the formulation is in the form of a gel, the pharmaceutical composition according to the invention also contains a gelling agent. The pharmaceutical composition in gel form according to the invention then has a content of between 0.05% and 3.0% of a gelling agent, preferably between 0.2% and 2% and even more preferably of 0.5%, these percentages being expressed by weight per 100 g of gel. Carbomers, cellulose derivatives, poloxamers, poloxamines or other gelling agents, alone or in combination, may be used in the formulation in the form of a gel according to the invention.

Carbomers are acrylic polymers. They may be used as suspension agents or to increase the viscosity of the gel formulations.

One carbomer that is particularly preferred in the context of the present invention is Carbopol® 980.

According to another advantageous embodiment of the pharmaceutical composition according to the invention when it is in the form of a gel and in the presence of certain types of gelling agent and preferably those containing carboxylic functions (—COOH) such as carbomers, it may contain a neutralizer. The neutralizer/gelling agent ratio is between 10/1 and 0.1/1, preferably between 7/1 and 0.5/1, and more preferentially it is 1/1. This neutralizer is chosen such that it forms, in the presence of the polymer, salts that are soluble in the solvent. The neutralizer is also chosen so as to be able to achieve optimum swelling of the polymer chains during the neutralization of the charges and the formation of polymer salts. According to the invention, triethanolamine is preferably used as a neutralizer in the presence of Carbopol® 980. It also makes it possible to achieve an optimum viscosity in the pharmaceutical composition according to the invention. Other neutralizers such as sodium hydroxide, ammonium hydroxide, potassium hydroxide, arginine, aminomethylpropanol or tromethamine may be used, alone or in combination, in preparation. The neutralizer is chosen as a function of the type of gelling agent used, in a manner which is known to those skilled in the art.

In general androgen therapy, the usual daily dose of the pharmaceutical composition in the form of a gel or a solution according to the invention is between 2.5 g and 5 g of the formulation per day.

The invention also relates to a process for preparing a pharmaceutical composition in the form of a gel or a solution according to the invention.

This process comprises the following successive steps:

DHT is dissolved, with stirring, in a mixture of solvents and absorption promoter;

water is added, with stirring, to the mixture obtained.

In the case of a gel, the following steps are added:

a gelling agent, such as carbopol, is then added to the mixture, with stirring;

optionally, a neutralizer such as triethanolamine is added to the mixture, with stirring.

The invention also relates to the use of the gel or the solution according to the invention for the preparation of a medicinal product for transdermal application for the treatment of a physiological condition associated with an androgen deficiency.

Examples of such physiological conditions which may be mentioned include:

in adults: permanent or functional hypogonadism with or without sexual dysfunction and/or with depression in men, hyperplasia of the prostate, gynaecomasty, balano-preputial sclero-atrophic lichen in men and vulval and perianal sclero-atrophic lichen in women;

in paediatrics: micropenis.

The pharmaceutical composition according to the invention may also comprise an oestrogen, preferably selected from the group consisting of 17β-oestradiol, oestrone, 17α-ethynyl-oestradiol and oestradiol valerianate, and even more preferably 17β-oestradiol at a dose bioequivalent to 0.5 mg of 17β-oestradiol administered orally.

DHT has an anti-gonadotropic and thus anti-oestrogen effect. Although it has not been proven that oestrogens play a positive role on the bones, it may be recommended, as a preventive measure, to combine the administration of oestrogen with that of DHT in men exhibiting insufficient level of oestradiole in the blood in order to compensate for this loss and to allow them to regain an acceptable physiological level.

The invention will be understood more clearly with the aid of the non-limiting examples described below.

### EXAMPLE 1

### Pharmaceutical Composition in the Form of a Gel According to the Invention

A gel according to the invention having the formulation below was prepared by the Applicant Company. The amounts are given per 100 g of gel:

| | | |
|---|---|---|
| Dihydrotestosterone | 0.7 | g |
| 95% Ethanol | 71.0 | g |
| Carbopol 980 | 0.5 | g |
| Isopropyl myristate | 0.7 | g |
| Triethanolamine | 0.5 | g |
| Purified water qs | 100.0 | g |

### EXAMPLE II

### Pharmaceutical Composition in the Form of a Solution According to the Invention

A solution according to the invention having the formulation below was prepared by the Applicant Company. The amounts are given per 100 g of solution:

| | | |
|---|---|---|
| Dihydrotestosterone | 0.7 | g |
| 95% Ethanol | 71.0 | g |
| Isopropyl myristate | 0.7 | g |
| Purified water qs | 100.0 | g |

### EXAMPLE III

### Process for Preparing a Gel According to the Invention

The manufacture of a DHT-based gel according to the invention is carried out as follows: for a batch of 70 kg containing 0.7% DHT, the following process is performed:

49 700 g of 95% ethanol are placed, under a vacuum of 800 mbar without stirring, in the tank of a mixer of Koruma™ type. Next, 490 g of isopropyl myristate are added via the top of the tank. Finally, 490 g of DHT are added via the top of the tank.

The above ingredients are mixed for 10 minutes, turbine at 2 000 rpm, doctor blade at 40 rpm, until the DHT is completely dissolved.

18 620 g of purified water are added under a vacuum of 800 mbar and mixing is carried out using a doctor blade at 40 rpm.

350 g of Carbopol® 980 are added under a vacuum of 800 mbar. Mixing is carried out at 2 000 rpm. The vacuum is broken. Mixing is carried out for 10 minutes, turbine at 2 000 rpm, doctor blade at 40 rpm.

Triethanolamine is added via the top of the tank. Mixing is carried out for 3 minutes, turbine at 2 000 rpm, doctor blade at 40 rpm.

The mixer is placed under a vacuum of 120 mbar for 2 to 3 minutes. Next, the vacuum is broken and stirring is then carried out for 20 minutes with the doctor blade at 40 rpm.

### EXAMPLE IV

### Process for Preparing a Solution According to the Invention

Manufacture of a DHT-based solution according to the invention is carried out as follows:

for a batch of 70 kg containing 0.7% DHT, the process is performed in the following manner:

49 700 g of 95% ethanol are added, under a vacuum of 800 mbar without stirring, to the tank of a mixer of Koruma™ type. Next, 490 g of isopropyl myristate are added via the top of the tank. Finally, 490 g of DHT are added via the top of the tank.

Mixing is carried out for 10 minutes, turbine at 2 000 rpm, doctor blade at 40 rpm, until the DHT has completely dissolved.

19 320 g of purified water are added under a vacuum of 800 mbar, doctor blade at 40 rpm.

The mixer is placed under a vacuum of 120 mbar for 2 to 3 minutes. Next, the vacuum is broken and stirring is then carried out for 20 minutes with the doctor blade at 40 rpm.

### EXAMPLE V

### Pharmacokinetic Studies In Vitro and In Vivo

In vitro Studies:

Comparative studies of percutaneous absorption on human abdominal skin between the formulation Andractim® containing 2.5% DHT and the 0.7% gel formulation described according to the invention were performed on Franz cells.

The formulations were applied at a dose of about 13 μg of DHT per cm² onto an area of 1.77 cm² of skin.

The results show that the levels of DHT absorbed are comparable between the two formulations: 0.47 μg and 0.32 μg of DHT absorbed, respectively, for the 0.7% gel and the 2.5% gel.

In vivo Study:

A phase I pharmacokinetic study was performed in order to compare the pharmacokinetic parameters of the formulation Andractim® containing 2.5% DHT and the formulation according to the invention containing 0.7% DHT after repeated percutaneous administration. This study was an open cross-over study on 18 patients without placebo. 5 g of Andractim® 2.5% gel (i.e. 125 mg of DHT) or 5 g of gel according to the invention at 0.7% (i.e. 35 mg of DHT) were administered once a day for 7 days.

The pharmacokinetic parameters of each of the formulations administered were evaluated and are as follows.

The average plasmatic concentrations of DHT observed as a function of the formulation used are summarized in the table below on days 1, 5 and 8 following the start of the study (day 0).

TABLE 1

| | Mean ± standard deviation (ng/mL) | | |
|---|---|---|---|
| | Day 1 | Day 5 | Day 8 |
| 2.5% Andractim | 0.597 ± 0.165 | 4.31 ± 2.06 | 3.62 ± 1.83 |
| 0.7% DHT gel | 0.621 ± 0.217 | 3.83 ± 2.02 | 3.38 ± 1.30 |

The AUCs (areas under the curve) calculated between 0 and 24 hours on day 7 are equal to 85.4 ng.h/mL (35% CV) after treatment with Andractim® 2.5% and 102 ng.h/mL (33% CV) after treatment with the DHT gel formulation according to the invention at 0.7%.

The average plasmatic concentrations of DHT on day 7 (from 0 to 24 hours) are equal to 3.98±1.32 ng/mL after treatment with Andractim® 2.5% and 4.60±1.51 ng/mL after treatment with the DHT gel formulation according to the invention at 0.7%.

The results obtained in vivo shows that the two treatments (Andractim® 2.5% and the DHT gel formulation according to the invention at 0.7%) show relatively similar pharmacokinetics.

The statistical tests performed in accordance with the international regulations in force for medicinal products intended to be administered to humans demonstrate that the bioequivalence is not significant between the two treatments and that the 0.7% DHT formulation according to the invention is superbioavailable relative to Andractim® 2.5%.

1. Pharmaceutical composition in the form of a gel or a solution, characterized in that it contains dihydrotestosterone and at least one percutaneous absorption promoter.

2. Pharmaceutical composition according to claim 1, with a dihydrotestosterone content of less than 2.5%, preferably less than 1.5%, and even more preferably of 0.7%, this percentage being expressed by weight relative to 100 g of formulation.

3. Pharmaceutical composition according to claim 1, with a solvent content of between 30% and 85%, preferably between 40% and 75%, and even more preferably of 71%, these percentages being expressed by weight relative to 100 g of formulation.

4. Pharmaceutical composition according to claim 3, in which the solvent is selected from the group consisting of ethanol, isopropanol and ethyl acetate, as well as mixtures thereof, and is preferably ethanol and/or isopropanol.

5. Pharmaceutical composition according to claim 1, with a content of between 0.1% and 10%, preferably between 0.3% and 5%, and even more preferably of 0.7% of a percutaneous absorption promoter, these percentages being expressed by weight relative to 100 g of formulation.

6. Pharmaceutical composition according to claim 5, in which the percutaneous absorption promoter is selected from the group consisting of aliphatic fatty acid esters such as isopropyl myristate; fatty acids such as oleic acid; alcohols or polyols such as ethanol, propylene glycol and polyethylene glycols; components of essential oils and terpine derivatives (such as eugenol, geraniol, nerol, eucalyptol or menthol); surfactants; moisturizers such as glycerol, urea; keratolytic agents such as α-hydroxy acids, and also mixtures thereof, and is preferably isopropyl myristate.

7. Pharmaceutical composition according to claim 1, with a content of between 0.05% and 3% of a gelling agent, preferably between 0.2% and 2% and even more preferably of 0.5%, these percentages being expressed by weight relative to 100 g of formulation.

8. Pharmaceutical composition according to claim 7, in which the gelling agent is selected from the group consisting of carbomers, cellulose derivatives, poloxamers, poloxamines and mixtures thereof, preferably carbomers and even more preferably being Carbopol® 980.

9. Pharmaceutical composition according to claim 7, further comprising a neutralizer.

10. Pharmaceutical composition according to claim 9, in which the neutralizer is selected from the group consisting of triethanolamine, sodium hydroxide, ammonium hydroxide, potassium hydroxide, arginine, aminomethylpropanol and tromethamine, and also mixtures thereof, and is preferably triethanolamine.

11. Pharmaceutical composition according to claim 9, in which the neutralizer/gelling agent ratio is between 10/1 and 0.1/1, preferably between 7/1 and 0.5/1, and even more preferably is 1/1.

12. Process for preparing a pharmaceutical composition according to claim 1, characterized in that:
a mixture composed of a solvent, an absorption promoter and DHT is prepared;
a gelling agent is optionally added to this mixture, and mixing is again carried out;
a neutralizer is optionally added, and mixing is then again carried out;
the pharmaceutical composition containing DHT is recovered.

13. Method for the treatment of physiological conditions associated with insufficiency of dihydrotestosterone, comprising the step of administering a pharmaceutical composition according to claim 1 to a subject.

14. Use according to claim 13, in which the physiological conditions are selected from the group consisting of permanent hypogonadism, functional hypogonadism, hyperplasia of the prostate, gynaecomasty, balano-preputial sclero-atrophic lichen in men and vulval sclero-atrophic lichen in women, or micropenis in children.
